(19) United States
(12) Patent Application Publication
(10) Pub. No.: US 2008/0244760 A1
(43) Pub. Date: Oct. 2, 2008

(54) Transgenic Frog Lines and Assays Employing Them

(75) Inventor: Nicolas Reed Marsh-Armstrong, Ellicott City, MD (US)

Correspondence Address: BANNER & WITCOFF, LTD., 1100 13th STREET, N.W., SUITE 1200, WASHINGTON, DC, 20005-4051, US

(73) Assignee: The John Hopkins University, Baltimore, MD (US)

(21) Appl. No.: 11/664,514

(22) PCT Filed: Oct. 3, 2005
(86) PCT No.: PCT/US05/35424

§ 371 Date: Jan. 9, 2008

Related U.S. Application Data
(60) Provisional application No. 60/615,855, filed on Oct. 4, 2004.

(51) Int. Cl. G01N 33/53; C12M 1/34; C12N 15/11; A01K 67/027

(52) U.S. Cl. 800/3; 435/287.2; 536/23.1; 800/13

(57) ABSTRACT

Rhodopsin transgenes driven by rhodopsin promoters were produced, some attached to GFP coding sequences as a fusion construct. When the resulting transgenes were introduced into Xenopus laevis, the photoreceptors degenerated in a manner similar to the degeneration observed in human retinal degenerations. Lines of animals with these transgenes were generated, and the progeny of such lines undergo similar patterns of degeneration. Since photoreceptors in these generated lines are marked by the expression of visible reporter proteins, and these reporters are visible and quantifiable externally through the lens of live animals, these lines may be used in the screening of therapeutics that prevent or slow photoreceptor degeneration.

This application claims the benefit of U.S. provisional application No. 60/615,855, filed Oct. 4, 2004, the disclosure of which is expressly incorporated herein.

### TECHNICAL FIELD OF THE INVENTION

The invention relates to transgenic frogs that can be used to screen for therapeutics that prevent or slow retinal degenerations. This invention also relates to the use of these transgenic frogs for screening test substances to identify those useful for preventing or slowing retinal degenerations.

### BACKGROUND OF INVENTION

Transgenesis refers to the process by which foreign nucleic acids are introduced into the genome of organisms. Transgenic organisms can be routinely made in many species of plants and animals. Many transgenic organisms have unique properties. For example, transgenic sheep and cows are capable of producing pharmaceuticals in their milk. Growth hormone transgenes have been introduced into livestock, such as cows and fish. Transgenic pigs that are more amenable to xenotransplantation procedures have also been produced. Transgenes can come from other organisms, species, genus, phylum, family, or even kingdom.

Transgenic frogs, of both Xenopus laevis and Silurana tropicalis, are generally made by restriction enzyme mediated nuclear transplantation (Amaya and Kroll, Methods Mol Biol. 97:393-414, 1999). This method allows the generation of over a hundred transgenic animals a day at a low cost, in which the transgenes typically integrate before the first cell division, and thus transgenic animals can be studied in the first generation without the need for generating transgenic lines. However, frog transgenic lines, which have been subjected to outcrossing and backcrossing, exhibit less variability of expression of transgenes relative to founder animals.

There is a need in the art for additional tools for screening for therapeutic agents and regimens that will be helpful for retinal degeneration. There is a need in the art for assays that can be scaled up for high through-put.

### SUMMARY OF INVENTION

According to one aspect of the invention a method is provided of screening for candidate therapeutic agents for treatment of retinal degeneration. A test substance is contacted with a first transgenic frog. The transgenic frog comprises a transgene which causes retinal degeneration and a reporter gene which encodes a fluorescent or luminescent protein or an enzyme which generates a fluorescent or luminescent product. Fluorescence or luminescence emitted from at least one eye of the first transgenic frog is detected. The emitted fluorescence or luminescence from the first transgenic frog is compared to that from a second transgenic frog which has not been contacted with the test substance. A higher emitted fluorescence or luminescence in the first transgenic frog than in the second transgenic frog indicates that the test substance is a candidate therapeutic agent for treatment of retinal degeneration.

According to another aspect of the invention a multi-well plate is provided. The plate is useful for assaying whole, live frog tadpoles. Each well contains a single frog tadpole.

Another aspect of the invention provides a genetic construct for assaying cells in a retina of a frog. The genetic construct comprises a reporter gene which encodes a fluorescent or luminescent protein or an enzyme which generates a fluorescent or luminescent product. The reporter gene is expressed in the retina under the control of a promoter which is active in frog photoreceptor cells.

Another embodiment of the invention is a transgenic frog which comprises a genetic construct. The genetic construct comprises a reporter gene which encodes a fluorescent or luminescent protein or an enzyme which generates a fluorescent or luminescent product. The reporter gene is expressed in the retina under the control of a promoter which is active in frog rod or cone cells. The transgenic frog may optionally be homozygous for the reporter gene. The transgenic frog may optionally comprise a transgene which causes retinal degeneration. The transgene may optionally be fused to the reporter gene.

These and other aspects and embodiments of the invention provide the art with tools and assays for performing high-throughput screening on whole, live animals. The tests can be performed non-invasively, making them low-cost and fast.

### BRIEF DESCRIPTION OF DRAWINGS

FIGS. 1A-B is the DNA and protein coding sequence of a bi-functional gene construct. (FIG. 1A) Schematic of a transgenic construct, which includes in a left to right direction, 5.5 kb of upstream promoter sequences from Xenopus rhodopsin gene (1), 3.5 kb of the rhodopsin gene locus (2), including introns (hatched), as well as 0.5 kb of downstream (3'UTR) sequences. The GFP cassette (3) was inserted in the last exon of rhodopsin. (FIG. 1B) Protein sequence has full-length wild-type Xenopus rhodopsin sequence (residue nos. 1-354) fused to a GFP sequence (residue nos. 368-605) through a linker sequence (residue nos. 355-367).

FIGS. 2A-G show that the bi-functional transgene induces and reports photoreceptor degeneration. (FIG. 2A) Fluorescence is visible through the lenses. (FIG. 2B) Low power view of retina without degeneration. (FIG. 2C) Low power view of retina with degeneration that shows a ventral preference; D-V, dorso-ventral axis. (FIG. 2D, FIG. 2F) Normal outer segments and protein localization in the same retina shown in B. (FIG. 2E, FIG. 2G) Degenerating outer segments and mislocalized rhodopsin in the same retina shown in FIG. 2C. Scale bars: 1 mm in FIG. 2A, 100 μm in FIG. 2 B-C, and 10 μm in FIG. 2D-G.

FIGS. 3A-C show that progeny from a transgenic line carrying a bi-functional construct can be used in an in vivo external assay to measure photoreceptor viability. (FIG. 3A) Measurements of eye fluorescent intensity in transgenic progeny and their control siblings, measured in live animals on a daily basis starting at 7 days of age. The fluorescence decreases to near baseline levels during the second week of life. (FIG. 3B) Typical retina section in transgenic animal at 7 days of age. (FIG. 3C) Typical retina section in transgenic animal at 16 days of age. Photoreceptors are lost from the central part of the retina, thus accounting for loss of fluorescence measured in FIG. 3A.

FIG. 4 shows an alignment of frog and human rhodopsin sequences with known human disease associated mutations indicated.

### DETAILED DESCRIPTION

It is a discovery of the present inventor that optically detectable reporter gene expression in the eyes of frogs can serve to monitor the status of the frogs' retinas. Notably, as the frog retina degenerates, expression of the optically detectable reporter gene decreases. Moreover, reporter genes are expressed very soon after hatching, thus making early detection possible. In addition, because of the small size of the whole organism at the time of initiation of detectable expression, assays can be performed in small volumes in multi-well format, facilitating high-throughput screens. Remarkably, despite different orientations of the whole organisms within the wells of a single plate, at least one eye of each organism is always optically accessible to a detection means on a single side of a multi-well plate.

### Degeneration-Inducing Gene Constructs.

Gene constructs based on the rhodopsin sequence have the potential to induce retinal degeneration when expressed in transgenic frogs. In one embodiment, the degeneration-inducing gene construct consists of a rhodopsin promoter sequence followed by a rhodopsin or rhodopsin variant protein coding sequence followed by poly-adenylation sequences. The promoter sequence used in many of our animals is a 5.5 kb upstream sequence from the Xenopus laevis rhodopsin gene. However other promoters that also drive expression in frog photoreceptors have been used and can be used interchangeably, provided they drive sufficiently high expression in frog photoreceptors. Promoter sequences can also be a smaller or larger fragment of the Xenopus rhodopsin gene, or from rhodopsin genes from Silurana tropicalis, Danio rerio or other fishes, or avian or mammalian species, including humans. Alternatively, the promoter may be the promoter of any other photoreceptor expressed gene from frogs or other species, such as cone opsin or cone arrestin. Alternatively, the promoter may be any promoter that drives transgene expression in frog photoreceptors; for example, the cytomegalovirus promoter or other viral promoters. The protein coding sequence of degeneration-inducing gene constructs may be rhodopsin or a rhodopsin variant and may be from any vertebrate species, including but not limited to Xenopus and human. Since all vertebrate rhodopsin protein coding sequences are highly conserved, they are expected to have similar function. Rhodopsin variants with substitutions in amino-acids that cause retinal degeneration when they are mutated can also be used. These “sensitive” amino-acids are particularly well conserved across vertebrate evolution. Thus, the same amino-acid substitutions are expected to have similar effects in the context of the rhodopsin sequence of various different vertebrate species. Though the majority of the constructs and animals made have been with either Xenopus or human cDNAs, the fact that both work equally well suggests that other untested vertebrate rhodopsin cDNA constructs will also work equally well. Similar constructs with other opsin sequences, for example, cone opsin sequences of various species, expressed in rod photoreceptors or cone photoreceptors, are expected to behave similarly and may be used interchangeably for this purpose. Alternatively, any other transgene that induces retinal degeneration in the frog, irrespective of the promoter or protein coding sequence, may be used.

### Degeneration-Reporting Gene Constructs.

Reporter gene constructs may comprise a promoter sequence, such as a rhodopsin promoter sequence or cone opsin promoter sequence, followed by a protein coding sequence of a reporter molecule. Optionally, these may be followed by poly-adenylation sequences. The rhodopsin promoter sequence may be from the Xenopus rhodopsin gene, or from rhodopsin genes from Silurana tropicalis, Danio rerio or other fishes, or avian or mammalian species, including humans. A cone-opsin promoter which may be used is that for the major cone cell type in Xenopus, or, alternatively, a red/green cone promoter from mammals or Danio rerio or other fish species or from avian species. Alternatively, the promoter may be the promoter of any other rod or cone photoreceptor-expressed genes from frogs or other species. Alternatively, the promoter may be a viral or synthetic promoter that drives transgene expression in frog photoreceptors; for example, the cytomegalovirus promoter or other viral promoters. The protein coding sequence of reporter gene constructs is a protein that can be used as an in vivo reporter, either through direct fluorescence or luminescence of the protein itself, or through an enzyme that has a substrate whose product is fluorescent or luminescent and that can be used in vivo. Among suitable proteins that are directly fluorescent which can be employed in the reporter constructs are the green fluorescent protein (GFP) and other fluorescent proteins derived from the jelly fish, Aqueous Victoria, or the fluorescent proteins from other marine species, and/or sequence variants of these fluorescent proteins; for example, EGFP, CFP, YFP, and DsRed can be used. Alternatively, the protein coding sequence of the reporter gene construct may be an enzyme, such as, beta-galactosidase, alkaline phosphatase, or other enzymes for which there is a fluorescent reporter substrate/product. Similarly, an enzyme such as luciferase from the firefly or other species that metabolizes a substrate into a product that is luminescent can be used. The reporter gene may encode a protein with any subcellular location, including cytoplasmic, membrane-associated, nuclear, or mitochondrial, or even secreted. One embodiment includes the localization of GFP or a GFP variant to the outer segments of photoreceptors, by means of transmembrane spanning regions, either synthetic or from natural transmembrane proteins, or by small targeting sequences such as those found in the C-terminus of rhodopsin proteins and retinol dehydrogenase.

Degeneration monitoring need not be related to a degeneration-causing transgene. Degeneration may be due to cultural conditions, such as light conditions, applied toxins or test substances, nutritional deprivations, etc. The reporter constructs of the present invention are thus useful for screening for agents which damage the retina, as well as for those which prevent degeneration.

### Bi-Functional Gene Constructs.

Bi-functional gene constructs encode both degeneration-inducing and degeneration-reporting functions in a single DNA construct. In one embodiment of the invention, the two degeneration-reporting and degeneration-inducing coding sequences are combined into a single, two promoter, linked construct. Alternatively, the two protein coding sequences are transcribed as a bicistronic messenger RNA that, through the function of an internal ribosome entry site (IRES), produces two separate functional proteins. In such constructs, either the degeneration-inducing coding sequence or the degeneration-reporting sequence may be the 3′ part of the construct, behind the IRES. Alternatively, in another embodiment of the invention, a single fusion protein functions as both the degeneration-inducing and degeneration-reporting mechanism. For example, a protein fusion construct can be made between a rhodopsin or rhodopsin variant protein coding sequence and a coding sequence of a fluorescent reporter protein, for example GFP. The reporter protein may be placed at the N- or C-terminus of the rhodopsin sequence, or internally within the rhodopsin sequence, for example within a cytoplasmic or luminal loop of the rhodopsin sequence, or alternatively within the C-terminal tail of rhodopsin, such that the localization and expression level of the fusion construct resembles the localization and expression of the endogenous rhodopsin protein. This reporter module, e.g., GFP, may be linked with or without linker sequences or other modifications that may promote better functionality at either inducing or reporting photoreceptor degeneration. These linked-constructs, bi-cistronic constructs, or fusion-constructs guarantee that any animal that is made transgenic for one construct, for example the degeneration-inducing construct, will necessarily express the other construct, for example the degeneration-reporting construct, and vice versa. These constructs may be made in the form of plasmid, cosmid or bacterial artificial chromosome (BAC) construct, or similar types of DNA molecules that are standard and known to those skilled in molecular biology.

Genomic and cDNA Constructs.

The constructs, whether separate degeneration-inducing and degeneration-reporting, or bi-functional constructs, may be made from either genomic or cDNAs. This distinction is principally relevant to the presence or absence of introns and other non-coding sequences. In one embodiment of the invention, the bi-functional construct is not made by adding a GFP cDNA to a rhodopsin cDNA, but rather by inserting the GFP cDNA within a larger DNA fragment that contains the frog rhodopsin genomic locus. Genomic transgenes are generally thought to promote higher levels of expression than cDNA transgenes. Both the ability to induce photoreceptor degeneration, as well as the ability to report that degeneration externally, require sufficiently high levels of transgene expression. Different constructs and even different animals with the same constructs are expected to produce different levels of transgene expression. Such variability is known in the field of transgenesis. Since animal models of mild as well as severe retinal degeneration are desirable for molecular screens of potential therapeutics, we have generated genomic and cDNA constructs, and hybrid genomic-cDNA constructs, that have different levels of expression.

### Substitutions, Additions and Deletions.

Many variations of the above designs are possible, such as various substitutions, additions, and deletions of promoter, protein coding and non-coding elements. For example, the exact position of GFP or other reporter molecule, the presence of absence of synthetic or natural introns, variants of different protein stability, and the like, are all foreseen in the current design.

Mutations in rhodopsin that have been identified in patients with autosomal dominant retinitis pigmentosum can be used. Some twenty-one of these are summarized in Sung et al., J. Biol. Chem. 268, 26645-26649, 1993. See Table 1. These include P23L, G51V, P53R, del 68-71, G106R, L125R, R135G, C167R, P171L, E181K, G182S, S186P, G188E, G188R, D190N, H211P, G211R, del 255, P167L, V345M, P347S. For other mutations which can be used see OMIM (Online Mendelial Inheritance in Man+180380.) See also P23H, (RetNet at rhodopsin), L125R, A164V, and G90D (EntrezGene for rhodopsin); and P347S, P347A, P347R, P347Q, P347L, P347T, V345M (Dikshit et al., J. Genet. 2001 August; 80(2):111-6.) Any mutations which are implicated in retinal degeneration can be used.

Transgenic Frogs with Degeneration-Inducing and Degeneration-Reporting Gene Constructs.

Transgenic frogs have been made transgenic with many of the constructs described in the previous sections. These animals were made transgenic by the REMI-nuclear transfer method devised by Amaya and Kroll (Methods Mol Biol. 97:393-414, 1999). Similar transgenic animals can be made by other methods, such as DNA injection into fertilized eggs with or without the assistance of transposase or other enzymatic activities. Any method, currently available and to be discovered in the future, that integrates the transgenic constructs into the genome of frogs can be used. The transgenes may integrate in single or multiple copies into single or multiple loci within the frog genomes. Outcrosses can be used to isolate a single insertion in the genome, i.e., in the hemizygous state. Backcrosses may be used to generate animals which carry two copies of a single insertion, i.e., that are homozygous for the insertion. The transgenes may be inserted into Xenopus laevis or Xenopus (Silurana) tropicalis genomes, or the genomes of other related frog species for which transgenic technologies are feasible. Transgenic animals can be selected by the expression of the reporter gene, or, alternatively, by the presence of a second fluorescent reporter transgene whose only function is to pre-select animals with a high probability of carrying the photoreceptor degeneration-inducing and/or degeneration-reporting construct. The presence of the photoreceptor degeneration-inducing and degeneration-reporting constructs can be verified by use of the polymerase chain reaction using primers that are complementary to sequences present in the transgenes but not complementary to frog genomic DNA. The expression level of the transgenes may be determined by use of mRNA or antibody probes. Degeneration of the frog retina can be determined in dissected eyes removed from live or sacrificed tadpoles, fixed, sectioned and analyzed histologically with common histological dyes and/or by use of antibodies. The reporter gene present in the degeneration-reporting construct can be used to determine whether there is degeneration in those transgenic animals and to determine the extent of such degeneration. Degeneration has been found to correlate well with the level of expression of the reporter gene.

Transgenic Frogs with Bi-Functional Gene Constructs.

Animals made with bi-functional gene constructs have a number of advantages over animals made with separate degeneration-inducing and degeneration-reporting gene constructs. The first is that, since the bi-functional constructs have both activities in a single construct, transgenic animals carrying both degeneration-inducing and degeneration-reporting activities can be determined unambiguously in live animals by visualizing the degeneration-reporting construct. For example, in the case that the degeneration-reporting activity is based on GFP fluorescence, transgenic animals can be separated from non-transgenic animals by simple and rapid visual inspection under a dissecting microscope equipped with epi-fluorescence. In this case, identification of transgenic animals, selection of animals with high, low or intermediate expression of transgenes, and selection of animals that undergo retinal degeneration can be determined in vivo by measurements of fluorescence emitted from the lens.

Lines of Frogs with Degeneration-Inducing and Degeneration-Reporting Gene Constructs.

Though transgenesis in frogs is a very low cost and efficient process, and large numbers of animals can be generated as primary transgenics, animal lines derived from primary transgenic founder animals have several advantages that can be exploited. Under the conditions in which we routinely make transgenic animals, the majority of the animals that are raised to sexual maturity breed and produce progeny that faithfully reproduce the transgene expression seen in the founder animal. In the majority of cases, lines are found to carry a single or two integration sites, as determined by approximately 50% or 75% of the progeny expressing the transgenes. Since frogs tend to produce over a thousand eggs in a single laying, the number of integration sites can be determined with a high degree of precision in a single breeding. Lines with a single integration site are particularly useful in this application, since different progeny have very similar levels of transgene expression and develop very similar phenotypes, in this case retinal degeneration. These single-integration lines are the preferred organisms with which to perform screening assays for identification of therapeutic agents and candidate therapeutic agents. Even in the case where a founder animal has a single integration site, if the animals were made with more than one transgene, the progeny tend to express the separate transgenes, suggesting that multiple transgenes integrate into the same locus, probably as concatamers. This is relevant to this application insofar as animals made with separate degeneration-inducing and degeneration-reporting constructs have a high probability of producing progeny that express both transgenes together.

### Sex of Founder Animals and Homozygosity of Transgenes.

Founder animals that are males have the advantage of a shorter generation time and the ability to produce progeny more frequently, and, thus, are preferred for drug screens (see below). Founder animals that are female are optimal for screening by transgenesis (see below). However, either males or females can be used for either type of screen. Transgenic animals in which the transgene loci have become homozygous by back-crosses or sibling-crosses can be used. These animals have the advantage of producing 100% of progeny with transgene expression and associated phenotypes, even when bred to non-transgenic animals. These homozygous animals, optimally those carrying single integration sites, are the preferred option for large-scale molecular screens.

Lines of Frogs with Bi-Functional Gene Constructs.

Lines of animals with bi-functional gene constructs that both produce retinal degeneration and report that degeneration are a preferred embodiment of this application. We have used some of these lines to demonstrate that the fluorescence measured externally through the lens is an accurate reporter for the degeneration that is occurring within the retina. The fluorescence measured externally, and the photoreceptor loss measured in sections, are highly consistent among different progeny carrying the same single integration sites. Different lines manifest different levels of transgene expression and different extents and speeds of retinal degenerations. These lines are ideal for screening therapeutics on a large-scale.

Screening Drugs with Photoreceptor-Degeneration Frog Lines

Photoreceptor degeneration frog lines are optimal for screening large numbers of chemical compounds. Individual frogs can produce thousands of progeny in a single mating. In some lines, photoreceptor degeneration occurs during the second week of life, all during a time when tadpoles can be raised individually in muti-well plates, such as 48-well or 96-well plates. Chemicals, or chemical libraries, can be added to the rearing water of these animals either manually or robotically. The fluorescence emitted through the lens can be detected by use of a fluorescence microscope or another type of automated fluorescence or luminescence detector. The animals can be imaged after being anesthetized or without anesthesia. The animals can be imaged at one time point, for example at a time point at which fluorescence is at the half-maximal level or down to baseline levels, or can be imaged repeatedly, for example on a daily basis. Imaging can be done advantageously in dark or opaque multi-well plates. Many variations of automation and quantification are foreseen to have different advantages and disadvantages, and may be applicable to different types of drug screens. Soluble drugs can be administered in the bath water. Insoluble drugs can be administered along with a vehicle such as nettle herb or another lipophilic food substance.

Optimizing Drugs with Photoreceptor-Degeneration Frog Lines.

The large number of animals that can be obtained from these lines enables the testing of multiple drug concentrations, multiple dosing regimens, as well as multiple drug formulations, purifications, and modifications, as well as the rapid retesting of existing or novel chemical derivatives of effective compounds. These lines also provide a test for whether drugs are metabolized, and the concentration at which various drugs begin to elicit side effects. These side effects can be determined by histological or molecular analyses of the animals, including potentially global analyses of gene expression or protein expression. These animals may also be subject to physiological analyses such as electro-retinograms (ERGs), electrocardiograms, and similar non-invasive procedures. ERGs will be particularly useful for monitoring the short and long-term effects of various drugs on photoreceptor viability. ERG analyses, for example, may be performed in young or old tadpoles, and in juvenile and adult frogs.

Screening cDNAs with Photoreceptor-Degeneration Frog Lines.

A major feature of these photoreceptor-degeneration frog lines derives from the fact that they are amenable to high-throughput transgenic screens. Transgenesis according to the present invention encompasses the introduction of a DNA construct into the genome of the frog, and the expression of the mRNA and proteins encoded by such DNA construct, usually under the control of ubiquitous or tissue-restricted or selective regulatory regions. Frog transgenesis is of such high efficiency and low cost that a single person can generate hundreds of distinct, primary, transgenic animals (founder animals) daily. Frog transgenesis is also amenable to scaling of many sorts, including automation of some of the most time-consuming aspects (see below). Thus, frogs are optimal for testing by transgenesis many gene products to determine whether they prevent or slow, or accelerate, retinal degeneration. The tested transgenes may be known or novel genes. By use of cell-type specific promoters, transgenes may be expressed selectively in different cell types: for example, in rod photoreceptors, cone photoreceptors, pigment epithelium, Müller cells, or other cells found inside or outside the retina, e.g., glandular organs such as the pituitary that can produce products and deliver them systemically.

### Robotic Injection.

Various steps of the transgenesis and screening procedure can be performed using automation. By using standard formats, such as 96-well plates, robotics developed for other purposes can be readily adapted to the present invention.

Screening Libraries of cDNAs with Photoreceptor Degeneration-Inducing Frog Lines.

A variant of screening cDNAs (as discussed above) is to screen libraries of cDNAs. These libraries may be collections of clones, for example all full-length cDNAs, or only full-length cDNAs of genes expressed in particular cell types, for example, in cones. Alternatively, these libraries may be a collection of unknown clones, for example those made from a cDNA library made from degenerating retinas, cone cell types, or the like. The clones can be pooled and screened together. In this “expression-cloning” strategy, once a pool of clones is identified with an effect, for example slowing retinal degeneration, the individual clones can be further fractionated until a single clone is identified. Clones present in animals with the desired biological effect can be determined by molecular methods, for example, by amplifying transgene inserts by PCR and identifying them by sequencing. These clones and libraries of clones can be expressed selectively in different cell types. For example, one can screen photoreceptor-expressed genes under the control of photoreceptor regulatory regions, and Müller cell-expressed genes under the control of Müller cell regulatory regions. Other permutations obvious to one in the field are also contemplated.

Screening Other Potential Therapeutics with Photoreceptor Degeneration-Inducing Frog Lines.

Above, we propose screening chemical compounds and cDNAs as trangenes. However, we anticipate using these lines to screen other types of substances and phenomena that may provide therapeutic benefit to degenerating retinas. These may include RNAs, coding and non-coding, for example micro-RNAs, as well as interfering constructs such as RNAi, either as trangenes or through other types of delivery, such as electroporation. Proteins and other molecules may be screened with suitable means of delivery. Natural substances can be screened. Physical conditions can be screened, such as light, radiation, temperature.

Xenopus laevis and Xenopus (Silurana) tropicalis.

The animal lines of the invention and their use may be either transgenic Xenopus laevis or Xenopus (Silurana) tropicalis. These highly related species are essentially interchangeable in terms of biology and techniques, such as transgenesis. Xenopus (Silurana) tropicalis has the advantage of a shorter generation time, a greater number of eggs produced, and a smaller embryo size. In addition, inbred lines of Silurana tropicalis are available and provide the advantage of displaying lower inter-animal variability. The smaller size of Xenopus (Silurana) tropicalis frogs and embryos will enable smaller set-ups for large-scale screens of therapeutics. The smaller size of eyes and lenses, as well as the higher maintenance requirement of Xenopus (Silurana) tropicalis are however disadvantages of this species. The same constructs, photoreceptor degeneration-inducing, degeneration-reporting or bi-functional constructs may be used in both species. Alternatively, species-specific promoters and/or protein coding sequences may be used. Should other frogs species become as amenable to transgenesis or drug screening applications, they too can be readily used to screen for therapeutics that prevent or slow retinal degeneration, or for agents which cause or accelerate degeneration.

### EXAMPLES

The following examples are provided by way of illustration only and not by way of limitation

### Example I

### Generation of Bi-Functional Gene Construct

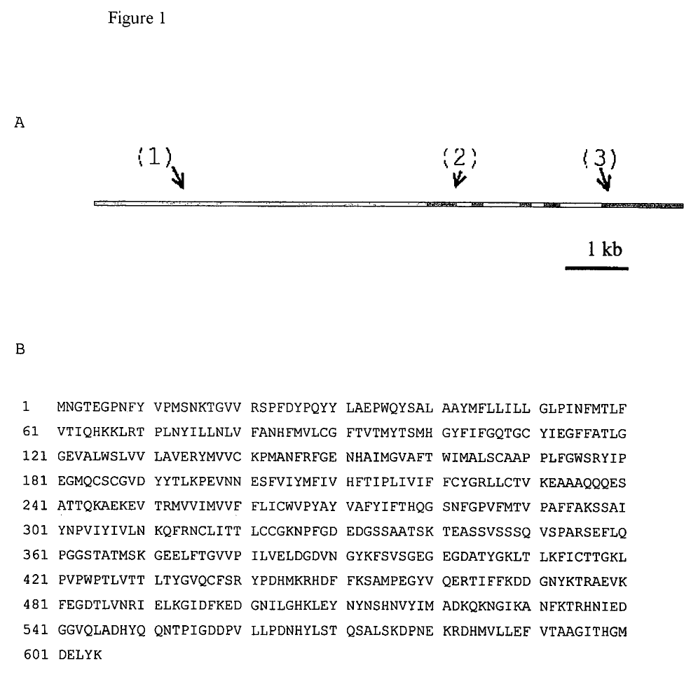

See FIG. 1 and brief description of same.

### Example II

### Generation of Transgenic Frogs with Bi-Functional Gene Construct

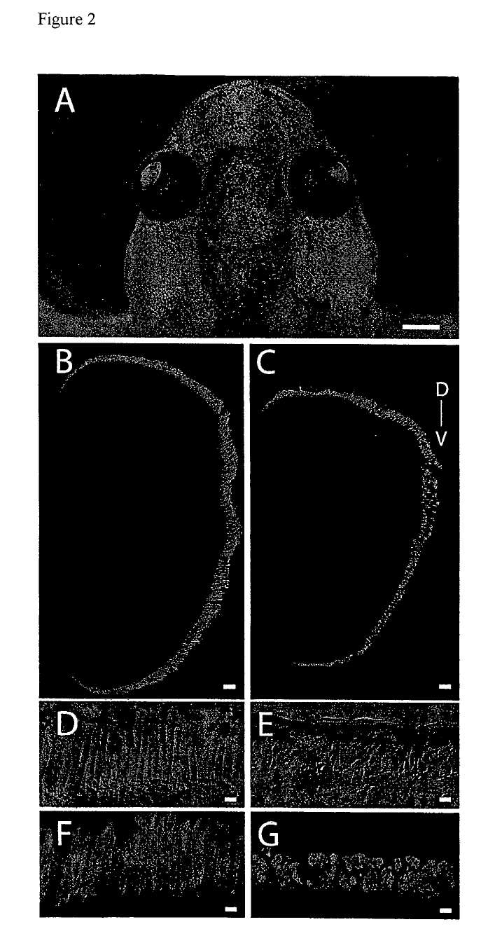

See FIG. 2 and brief description of same.

### Example III

### Measuring Photoreceptor Degeneration Externally in Live Tadpoles

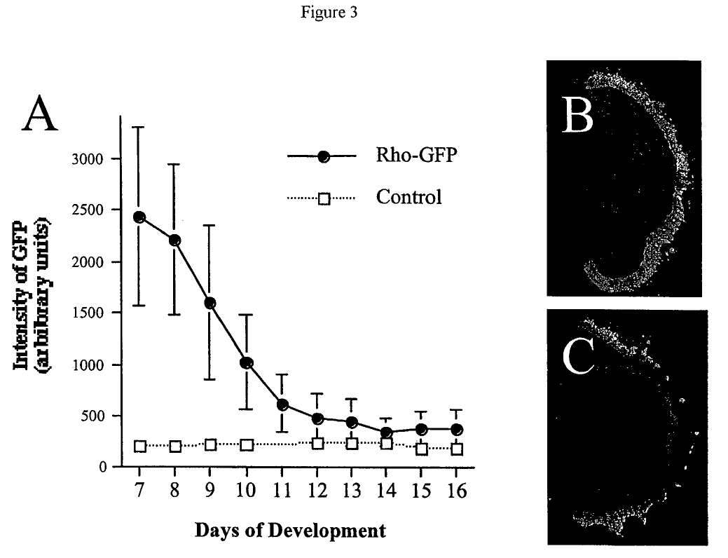

See FIG. 3 and brief description of same.

### Example IV

Transgenes were produced which have either the frog rhodopsin cDNA driven by the frog rhodopsin promoter, a frog rhodopsin genomic fragment (with introns) including the rhodopsin promoter, either as wild-type (normal) versions or mutated versions, carrying mutations known to cause autosomal dominant retinitis pigmentosa in humans. Some of these transgenes had GFP present as a fusion construct either at or near the C-terminus of the rhodopsin sequence. These constructs were made either by restriction enzyme based standard cloning procedures or by recombination based cloning procedures.

These constructs were used to generate transgenic frogs. The rhodopsin transgenes expressed in photoreceptors cause the photoreceptors to degenerate. The GFP transgenes, also expressed in photoreceptors, report the viability of the photoreceptors.

Transgenic frogs were raised and used to generate transgenic lines. In these lines, the fluorescence measured through the lens is an accurate and sensitive reporter for the viability of photoreceptors within the animal. These lines enable easy and large-scale screens for therapeutics that aim to prevent or slow retinal degeneration. Such therapeutics are useful in the treatment of retinal degenerations including Retinitis Pigmentosa, Macular degeneration, and other types of genetic and sporadic cone and rod dystrophies.

I claim:

1. A method of screening for candidate therapeutic agents for treatment of retinal degeneration, comprising:
contacting a test substance with a first transgenic frog, wherein the transgenic frog comprises a transgene which causes retinal degeneration and a reporter gene which encodes a fluorescent or luminescent protein or an enzyme which generates a fluorescent or luminescent product;
detecting fluorescence or luminescence emitted from at least one eye of the first transgenic frog;
comparing the emitted fluorescence or luminescence from the first transgenic frog to that from a second transgenic frog which has not been contacted with the test substance, wherein a higher emitted fluorescence or luminescence in the first transgenic frog than in the second transgenic frog indicates that the test substance is a candidate therapeutic agent for treatment of retinal degeneration.

2. The method of claim 1 wherein the transgene encodes a rhodopsin.

3. The method of claim 1 wherein the transgene encodes a frog rhodopsin.

4. The method of claim 1 wherein the transgene encodes an human rhodopsin.

5. The method of claim 1 wherein the transgene encodes a wild-type rhodopsin.

6. The method of claim 1 wherein the transgene encodes a wild-type frog rhodopsin.

7. The method of claim 1 wherein the transgene encodes a wild-type human rhodopsin.

8. The method of claim 1 wherein the transgene encodes a human rhodopsin with a mutation which causes retinal degeneration in humans.

9. The method of claim 1 wherein the transgene is a cDNA construct.

10. The method of claim 1 wherein the transgene is a genomic construct.

11. The method of claim 1 wherein the reporter gene encodes a fluorescent protein and the first and second frogs are illuminated using light at an excitatory wavelength for the fluorescent protein.

12. The method of claim 1 wherein the fluorescent or luminescent protein is GFP from Aqueous Victoria.

13. The method of claim 1 wherein the fluorescent or luminescent protein is selected from the group consisting of EGFP, CFP, YFP, and DsRed.

14. The method of claim 1 wherein the first and second frogs are tadpoles.

15. The method of claim 1 wherein the first and second frogs are in multi-well plates.

16. The method of claim 1 wherein the first and second transgenic frogs are homozygous for the transgene.

17. The method of claim 1 wherein the first and second transgenic frogs are homozygous for the transgene and for the reporter gene.

18. The method of claim 1 wherein the fluorescent or luminescent protein or the enzyme is expressed as a fusion protein with a protein encoded by the transgene in the first and second transgenic frogs.

19. The method of claim 18 wherein the first and second transgenic frogs are homozygous for the fusion protein.

20. A multi-well plate for assaying whole, live, transgenic frog tadpoles, wherein each well contains a single frog tadpole.

21. The multi-well plate of claim 20 wherein each frog tadpole comprises a first transgene.

22. The multi-well plate of claim 20 wherein the frog tadpole is a Xenopus laevis.

23. The multi-well plate of claim 20 wherein the frog tadpole is a Silurana tropicalis.

24. The multi-well plate of claim 20 wherein each frog tadpole comprises a reporter gene which encodes a fluorescent or luminescent protein or an enzyme which generates a fluorescent or luminescent product, wherein the reporter gene is expressed in retinas of the frog.

25. The multi-well plate of claim 20 wherein the plate is opaque.

26. A genetic construct for assaying cells in a retina of a frog, comprising:
a genomic DNA encoding a first protein, and
a reporter gene encoding a second protein which is fluorescent or luminescent or which is an enzyme which generates a fluorescent or luminescent product, wherein the genomic DNA and the reporter gene are expressed in the retina under the control of one or more promoters which are active in frog photoreceptor cells.

27. The genetic construct of claim 26 wherein the reporter gene encodes GFP.

28. The genetic construct of claim 26 wherein the one or more promoters are active in frog rod photoreceptors.

29. The genetic construct of claim 26 wherein the one or more promoters are active in frog cone photoreceptors.

30. The genetic construct of claim 26 wherein the one or more promoters comprise a rhodopsin promoter.

31. The genetic construct of claim 26 wherein the one or more promoters comprise a cone opsin promoter.

32. The genetic construct of claim 26 wherein the one or more promoters comprise a cone arrestin promoter.

33. The genetic construct of claim 26 wherein the reporter gene encodes a fluorescent protein selected from the group consisting of EGFP, CFP, YFP, and DsRed.

34. The genetic construct of claim 26 wherein the fluorescent or luminescent protein or the enzyme is fused to the first protein, and the first protein causes retinal degeneration.

35. The genetic construct of claim 26 wherein the fluorescent or luminescent protein or the enzyme is fused to the first protein and the first protein causes retinal degeneration, and wherein the first protein is amino-terminal with respect to the fluorescent or luminescent protein or the enzyme.

36. The genetic construct of claim 26 wherein the first protein causes retinal degeneration, and wherein the reporter gene and the genomic DNA encode a bi-cistronic message.

37. The genetic construct of claim 26 wherein the fluorescent or luminescent protein or the enzyme is fused to a rhodopsin protein.

38. The genetic construct of claim 37 wherein the rhodopsin protein is wild-type human rhodopsin.

39. The genetic construct of claim 37 wherein the rhodopsin protein is human rhodopsin that carries a mutation that causes retinal degeneration in humans.

40. The genetic construct of claim 37 wherein the rhodopsin protein is frog rhodopsin.

41. The genetic construct of claim 37 wherein the rhodopsin protein is wild-type frog rhodopsin.

42. The genetic construct of claim 34 wherein the protein which causes retinal degeneration is encoded in the genetic construct by genomic DNA.

43. A transgenic frog which comprises a genetic construct according to claim 26.

44. The transgenic frog of claim 43 which is homozygous for the reporter gene.

45. A transgenic frog which comprises a genetic construct according to claim 34.

46. The transgenic frog of claim 45 which is homozygous for the reporter gene.

47. A transgenic frog which comprises a genetic construct according to claim 36.

48. The transgenic frog of claim 47 which is homozygous for the reporter gene.


SEQUENCE LISTING (1 sequence)

SEQ ID NO: 1 - protein, 605 aa, Xenopus laevis
MNGTEGPNFYVPMSNKTGVVRSPFDYPQYYLAEPWQYSALAAYMFLLILLGLPINFMTLFVTIQHKKLRTPLNYILLNLVFANHFMVLCGFTVTMYTSMHGYFIFGQTGCYIEGFFATLGGEVALWSLVVLAVERYMVVCKPMANFRFGENHAIMGVAFTWIMALSCAAPPLFGWSRYIPEGMQCSCGVDYYTLKPEVNNESFVIYMFIVHFTIPLIVIFFCYGRLLCTVKEAAAQQQESATTQKAEKEVTRMVVIMVVFFLICWVPYAYVAFYIFTHQGSNFGPVFMTVPAFFAKSSAIYNPVIYIVLNKQFRNCLITTLCCGKNPFGDEDGSSAATSKTEASSVSSSQVSPARSEFLQPGGSTATMSKGEELFTGVVPILVELDGDVNGYKFSVSGEGEGDATYGKLTLKFICTTGKLPVPWPTLVTTLTYGVQCFSRYPDHMKRHDFFKSAMPEGYVQERTIFFKDDGNYKTRAEVKFEGDTLVNRIELKGIDFKEDGNILGHKLEYNYNSHNVYIMADKQKNGIKANFKTRHNIEDGGVQLADHYQQNTPIGDDPVLLPDNHYLSTQSALSKDPNEKRDHMVLLEFVTAAGITHGMDELYK